(19) United States
(12) Patent Application Publication
(10) Pub. No.: US 2024/0335401 A1
(43) Pub. Date: Oct. 10, 2024

(54) SUBSTITUTED PHENETHYLAMINE FOR TREATING INFLAMMATION AND PSYCHOLOGICAL DISORDERS

(71) Applicant: SYNAPTIVE THERAPEUTICS LLC, PEARLAND, TX (US)

(72) Inventor: Matthew Halpert, Pearland, TX (US)

(21) Appl. No.: 18/572,752

(22) PCT Filed: Jun. 23, 2022
(86) PCT No.: PCT/US2022/034778

§ 371 Date: Dec. 20, 2023

(51) Int. Cl. A61K 31/137; A61K 31/133; A61K 31/145; A61K 31/15; A61K 31/343; A61K 31/357; A61K 31/402; A61K 31/4402; A61K 31/4458; A61K 31/451; A61K 31/5375; A61P 29/00

(52) U.S. Cl. CPC A61K 31/137; A61K 31/133; A61K 31/145; A61K 31/15; A61K 31/343; A61K 31/357; A61K 31/402; A61K 31/4402; A61K 31/4458; A61K 31/451; A61K 31/5375; A61P 29/00

(57) ABSTRACT

Compositions comprising a substituted phenethylamine and methods of using the compositions for treating an inflammatory or neurological disorder in a subject in need thereof are disclosed. The substituted phenethylamine can be used at sub-hallucinogenic concentrations.

### CROSS-REFERENCE TO RELATED APPLICATIONS

The present application is a National Stage application of International Application No. PCT/US22/34778, filed Jun. 23, 2022, and published as WO 2022/271982 on Dec. 29, 2022 claims priority from Provisional Application No. 63/214,129, filed Jun. 23, 2021, and Provisional Application No. 63/224,163, filed Jul. 21, 2021, the entire contents of each of which is hereby incorporated by reference.

### FIELD OF THE INVENTION

The present disclosure provides compositions comprising a substituted phenethylamine and methods of using the compositions for treating an inflammatory or neurological disorder.

### BACKGROUND OF THE INVENTION

Inflammation. Acute inflammation occurs in response to an injury such as cuts or bruises. In acute inflammation, the immune system releases white blood cells to surround and protect the injured area. This results in the familiar redness, warmth, swelling, and pain that surrounds tissue and joints that occurs in response to injuries, such as cuts and bruises. The immune system responds in a somewhat similar manner when a person is infected with a pathogen such as a virus like a cold or the flu. In extreme cases, however, such as during sepsis or more recently, in persons infected with the coronavirus COVID-19, the immune system overwhelms the body leading some to experience organ failure, septic shock and death. At least 1.7 million adults in the U.S. develop sepsis, and nearly 270,000 die as a result. It has been reported that 1 in 3 patients that die in a hospital, die of sepsis. An estimated 11 million people die each year from sepsis, worldwide. However, attempts at developing effective treatment for extreme cases of acute inflammation such as sepsis have not been successful.

Chronic systemic inflammation can contribute to the development or progression of certain conditions such as cardiovascular disease, cancer, diabetes mellitus, chronic kidney disease, non-alcoholic fatty liver disease, allergies, autoimmune and neurodegenerative disorders, coronary heart disease, and chronic obstructive pulmonary disease (COPD) are examples of diseases mediated by chronic inflammation. Obesity, smoking, stress, and insufficient diet are some of the factors that promote chronic inflammation. A 2014 study reported that 60% of Americans had at least one chronic inflammatory condition, and 42% had more than one. Treatments for chronic inflammation and conditions associated with chronic inflammation include non-steroidal anti-inflammatory drugs (NSAIDs), corticosteroids, and serotonins. However, none of these treatments can effectively and safely treat chronic inflammation and disorders associated with chronic inflammation. For instance, NSAIDS can alleviate the pain caused by chronic inflammation in patients with arthritis, however, persons taking NSAIDs often experience one or more side effects including but not limited to gas, feeling bloated, heartburn, stomach pain, nausea, vomiting, diarrhea and/or constipation. Further, long term use of NSAIDs could harm the microbiome in the gut causing inflammation in intestinal walls, a condition known as leaky gut, which in turn releases toxins and triggers chronic bodywide inflammation. In addition, it is not uncommon for a person to develop stomach ulcers attributed to the use of NSAIDs.

Accordingly, there is a recognized and urgent need for effective and safe treatments for inflammation and disorders associated with inflammation.

Psychological disorders. It has been reported that about one in five American adults, an alarming 20% of the adult U.S. population, suffers from some form of mental illness or psychological disorder. It has also been reported that nearly half of the U.S. population, i.e., 46%, will have a diagnosable mental health condition at some point in their life, and that nearly half of those persons will develop these conditions by the age of fourteen. Diagnosed mental health conditions include a variety of psychological disorders including anxiety, addiction/substance abuse, bipolar disorder, various forms of depression, post-traumatic stress disorder, more commonly known as PTSD, schizophrenia, and suicidal thoughts among others. In more severe cases, a person may experience combinations of several of these psychological disorders. Treatment of psychological disorders currently consist of medication regimens, therapy sessions, or combinations of the two. Some of the more common types of prescribed medications include, but are not limited to, antianxiety medications; antidepressant medications to improve moods; antipsychotic medications to treat disordered thought patterns and altered perceptions, and/or mood-stabilizing medications. Given the wide range of medications typically prescribed for various psychological disorders, the efficacy of a particular medication for a particular disorder for a particular patient is highly variable. Often, identifying a safe and effective treatment for each patient is a hit or miss strategy depending on a patient's reaction to a particular medication or dosing regimen, which can result in a patient being over, under or improperly treated for potentially extended periods of time causing unnecessary discomfort, and perhaps worse.

In addition, there are a wide range of side effects, from mild to severe, which have been observed in patients taking these various medications. As one example, a common class of antidepressants include selective serotonin reuptake inhibitors, or SSRIs. Numerous negative side effects are associated with SSRIs including, but not limited to: feeling agitated, shaky or anxious; feelings of being sick; indigestion; diarrhea or constipation; loss of appetite and weight loss; dizziness; blurred vision; dry mouth; excessive sweating; sleeping problems, including insomnia or drowsiness; headaches; reduced sex drive; difficulty achieving orgasm during sex or masturbation; and in men, difficulty obtaining or maintaining an erection; i.e., erectile dysfunction. Serotonin-norepinephrine reuptake inhibitors, or SNRIs, are another class of antidepressants that are often prescribed to treat depression as well as anxiety. Common side effects associated with SNRIs essentially parallels the side effects noted above for SSRIs. Other classes of medications often prescribed to treat persons with psychological disorders include tricyclic antidepressants and benzodiazepines, each coming with their own set of similar negative side effects, with the addition of potential addiction and/or overdose in the case of benzodiazepines.

Accordingly, there is an established need for a solution to the foregoing problems and shortcomings in the present state of the art with regard to methods of treatment for psychological disorders with medication.

### SUMMARY OF THE INVENTION

In one aspect, the instant disclosure encompasses a method of treating an inflammatory or neurological disorder in a subject in need thereof. The method comprises administering to the subject a therapeutically effective amount of a composition comprising a substituted phenethylamine. Administering the composition can comprise administering a unit dose of the composition to the subject, wherein the unit dose comprises a therapeutically effective amount of the substituted phenethylamine. In some aspects, the substituted phenethylamine is administered at a sub-hallucinogenic concentration.

The substituted phenethylamine can be a substituted phenethylamine of Table 1. In some aspects, the substituted phenethylamine is a member of the 2C-x family of substituted phenethylamines. In some aspects, the substituted phenethylamine is a member of the 2C-x family of substituted phenethylamines of Table 2. For instance, the substituted phenethylamine is 2C-H, 2C-I, 2C-B, or 2C-E.

The method can be used to treat inflammation. The inflammation can be chronic inflammation or acute inflammation. When the method is used to treat inflammation, the substituted phenethylamine can be 2C-H or 2C-I. The inflammation can be chronic inflammation and the composition can be administered in a unit dose comprising 2C-H at a concentration ranging from about 1 mg to about 100 mg, at a concentration of about 25 mg, at a concentration of about 50 mg, or at a concentration of about 100 mg. In other aspects, the inflammation is mild chronic inflammation and the composition is administered in a unit dose comprising 2C-H at a concentration of about 25 mg. In yet other aspects the inflammation is moderate chronic inflammation and the composition is administered in a unit dose comprising 2C-H at a concentration of about 50 mg. In additional aspects, the inflammation is severe chronic inflammation and the composition is administered in a unit dose comprising 2C-H at a concentration of about 100 mg.

The inflammation can be acute inflammation and the composition can be administered in a unit dose comprising 2C-H at a concentration ranging from about 1 mg to about 100 mg, at a concentration ranging from about 100 mg to about 200 mg, at a concentration of about 25 mg, at a concentration of about 50 mg, at a concentration of about 100 mg, at a concentration of about 150 mg, or at a concentration of about 200 mg. In some aspects, the inflammation is mild acute inflammation and the composition is administered in a unit dose comprising 2C-H at a concentration of about 25 mg. In other aspects, the inflammation is moderate acute inflammation and the composition is administered in a unit dose comprising 2C-H at a concentration of about 50 mg. In additional aspects, the inflammation is severe acute inflammation and the composition is administered in a unit dose comprising 2C-H at a concentration of about 100 mg. In yet other aspects, the inflammation is systemic acute inflammation and the composition is administered in a unit dose comprising 2C-H at a concentration of about 150 mg or at a concentration of about 200 mg.

The therapeutically effective amount of 2C-H can range from about 0.1 mg/kg to about 1 mg/kg body weight of the subject.

The method can also be used to treat a psychological disorder by administering a therapeutically effective amount of substituted phenethylamine. The substituted phenethylamine can be 2C-B or 2C-E. In some aspects, the composition is administered in a unit dose comprising 2C-B at a concentration ranging from about 1 mg to about 10 mg, from about 1 mg to about 20 mg, from about 1 mg to about 100 mg, at about 10 mg, at about 15 mg, at about 20 mg, or at about 25 mg. In some aspects, the therapeutically effective amount of 2C-B ranges from about 0.1 mg/kg to about 1 mg/kg body weight of the subject.

In another aspect, the instant disclosure encompasses a pharmaceutical composition for treating inflammation or a neurological disorder. The composition comprises a substituted phenethylamine. The substituted phenethylamine can be a substituted phenethylamine of Table 1.

In some aspects, the substituted phenethylamine is a member of the 2C-x family of substituted phenethylamines. For instance, the substituted phenethylamine can be a member of the 2C-x family of substituted phenethylamines of Table 2. The substituted phenethylamine can be 2C-H, 2C-I, 2C-B, or 2C-E. In some aspects, the substituted phenethylamine is 2C-H. The composition can be in the form of a unit dose comprising 2C-H at a concentration ranging from about 1 mg to about 100 mg, at a concentration ranging from about 100 mg to about 200 mg, at a concentration of about 25 mg, at a concentration of about 50 mg, at a concentration of about 100 mg, at a concentration of about 150 mg, or at a concentration of about 200 mg.

In some aspects, the substituted phenethylamine is 2C-B. The composition can be in the form of a unit dose comprising a sub-hallucinogenic dose of 2C-B. In some aspects, the composition is in the form of a unit dose comprising 2C-B at a concentration ranging from about 1 mg to about 10 mg, from about 1 mg to about 20 mg, from about 1 mg to about 100 mg, at about 10 mg, at about 15 mg, at about 20 mg, or at about 25 mg.

Yet another aspect of the instant disclosure encompasses a kit for comprising one or more pharmaceutical compositions comprising a substituted phenethylamine. The compositions can be as described herein above.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a graph showing the level of TNF-α in serum of mice injected with LPS to induce inflammation and administered 2C-H or control vehicle.

FIG. 2 is a plot showing % mice that died after injection of a toxic level of LPS when administered 2C-H or vehicle control.

FIG. 3 is a plot of % mice showing sign of hallucination at increasing doses of 2C-B.

FIG. 4 is a graph showing the time spent on the open arm by mice treated with 2C-B for increasing amounts of time.

### DETAILED DESCRIPTION

The present disclosure is based in part on the surprising discovery that substituted phenethylamines, can treat inflammation and psychological disorders. Importantly, the compositions and methods of the instant disclosure can treat inflammation and a wide range of inflammatory and psychological disorders that currently must be treated using a hit or miss approach. Additionally, when a substituted phenethylamine is a compound that exhibits psychoactive activity, a composition of the instant disclosure can be effective at treating inflammation and psychological disorders even when administered at sub-psychoactive doses or concentrations.

### I. Composition

One aspect of the present disclosure encompasses pharmaceutical compositions comprising a substituted phenethylamine for treating inflammation and disorders associated with inflammation, and psychological disorders.

### (a) Substituted Phenethylamines

Substituted phenethylamines (phenethylamines) are organic compounds based on the phenethylamine structure shown below. The class comprises all the derivative compounds of phenethylamine which can be formed by replacing, or substituting, one or more hydrogen atoms in the phenethylamine core structure with substituents.

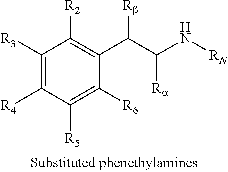

Many substituted phenethylamines are psychoactive drugs belonging to a variety of different drug classes, including central nervous system stimulants (e.g., amphetamine), hallucinogens (e.g., dl-2,5-dimethoxy-4-methylamphetamine a.k.a. DOM), entactogens (e.g., 3,4-methylenedioxyamphetamine a.k.a. MDA), appetite suppressants (e.g. phentermine), nasal decongestants and bronchodilators (e.g., levomethamphetamine and pseudoephedrine), antidepressants (e.g. bupropion and phenelzine), anti-parkinson agents (e.g., selegiline), and vasopressors (e.g., ephedrine), among others. Numerous endogenous compounds—including hormones, catecholamines such as dopamine and noradrenaline, and many trace amines (e.g. adrenaline, phenethylamine itself, tyramine, thyronamine, and iodothyronamine)—are substituted phenethylamines. Several notable recreational drugs, such as MDMA (ecstasy), methamphetamine, and cathinone, are also members of the class. All the substituted amphetamines and substituted methylenedioxyphenethylamines are substituted phenethylamines as well. Many of these psychoactive compounds exert their pharmacological effects primarily by modulating monoamine neurotransmitter systems; however, there is no mechanism of action or biological target that is common to all members of this subclass. Non-limiting examples of substituted phenethylamines and their biologic activity is shown in Table 1.

TABLE 1
Selected substituted phenethylamines

| Short Name | R^{N} | R^{α} | R^{β} | R² | R³ | R⁴ | R⁵ | Biologic activity |
|---|---|---|---|---|---|---|---|---|
| meta-Tyramine | | | | | OH | | | Trace amine |
| para-Tyramine | | | | | | OH | | Trace amine |
| Dopamine | | | | | OH | OH | | Catecholamine |
| | | | | | | | | neurotransmitter |
| Epinephrine | CH₃ | | OH | | OH | OH | | Catecholamine |
| (Adrenaline) | | | | | | | | neurotransmitter/ |
| | | | | | | | | Fight or Flight |
| | | | | | | | | hormone |
| Norepinephrine | | | OH | | OH | OH | | Catecholamine |
| (Noradrenaline) | | | | | | | | neurotransmitter/ |
| | | | | | | | | Fight or Flight |
| | | | | | | | | hormone |
| meta-Octopamine | | | OH | | OH | | | Trace amine |
| para-Octopamine | | | OH | | | OH | | Trace aminergic |
| | | | | | | | | α-adrenoceptor |
| | | | | | | | | agonist |
| Phenylephrine | CH₃ | | OH | | OH | | | α-adrenergic |
| | | | | | | | | agonist; |
| | | | | | | | | decongestant |
| 6- | | | | OH | | OH | OH | neurotoxic agent |
| Hydroxydopamine | | | | | | | | for the dopamine |
| | | | | | | | | and |
| | | | | | | | | norepinephrine |
| | | | | | | | | receptors |
| Salbutamol | C(CH₃)₃ | | OH | | | OH | CH₂OH | Short-action β2- |
| | | | | | | | | adrenergic |
| | | | | | | | | agonist |
| β- | | | CH₃ | | | | | Stimulant |
| Methylphenethylamine | | | | | | | | |
| Amphetamine | | CH₃ | | | | | | Monoamine |
| | | | | | | | | releasing agent; |
| | | | | | | | | Stimulant |
| N- | CH₃ | | | | | | | Trace amine; |
| Methylphenethylamine | | | | | | | | endogenous |
| | | | | | | | | amphetamine |
| | | | | | | | | isomer |
| N,N- | (CH₃)₂ | | | | | | | Trivial effects |
| Dimethylphenethylamine | | | | | | | | (used as a food |
| | | | | | | | | additive and |
| | | | | | | | | flavoring agent) |
| Methamphetamine | CH₃ | CH₃ | | | | | | Monoamine |
| | | | | | | | | releasing agent; |
| | | | | | | | | stimulant; |
| | | | | | | | | neurotoxin |
| Phentermine | | (CH₃)₂ | | | | | | Stimulant, |
| | | | | | | | | anorectic |
| Ortetamine | | CH₃ | | CH₃ | | | | Stimulant, |
| | | | | | | | | anorectic |
| Phenelzine | NH₂ | | | | | | | Monoamine |
| | | | | | | | | oxidaseinhibitor |
| Methylphenidate | —CH₂—CH₂—CH₂—CH₂— | | C(OCH₃)═O | | | | | NDRI; Stimulant |
| Ephedrine/ | CH₃ | CH₃ | OH | | | | | Releasing agent; |
| Pseudoephedrine | | | | | | | | stimulant; |
| | | | | | | | | decongestant |
| Cathine | | CH₃ | OH | | | | | Moderately |
| | | | | | | | | selective |
| | | | | | | | | norepinephrine- |
| | | | | | | | | releasing agent |
| Cathinone | | CH₃ | ═O | | | | | Selective |
| | | | | | | | | norepinephrine |
| | | | | | | | | and dopamine |
| | | | | | | | | releasing agent |
| Methcathinone | CH₃ | CH₃ | ═O | | | | | Selective |
| | | | | | | | | norepinephrine |
| | | | | | | | | and dopamine |
| | | | | | | | | releasing agent |
| Mephedrone | CH₃ | CH₃ | ═O | | | CH₃ | | Stimulant, |
| | | | | | | | | unknown |
| | | | | | | | | pharmacodynamic |
| | | | | | | | | actions |
| Ethcathinone | CH₂CH₃ | CH₃ | ═O | | | | | Stimulant and |
| | | | | | | | | norepinephrine |
| | | | | | | | | releasing agent |
| Bupropion | C(CH₃)₃ | CH₃ | ═O | | Cl | | | NDRI |
| Norfenfluramine | | CH₃ | | | CF₃ | | | SSRA |
| Fenfluramine | CH₂CH₃ | CH₃ | | | CF₃ | | | SSRA |
| 5-APB | | CH₃ | | | —CH═CH—O— | | | Stimulant, |
| | | | | | | | | entactogen |
| 6-APB | | CH₃ | | | —O—CH═CH— | | | Stimulant, |
| | | | | | | | | entactogen |
| MDA | | CH₃ | | | —O—CH₂—O— | | | Stimulant, |
| | | | | | | | | psychedelic, |
| | | | | | | | | entactogen |
| MDEA | CH₂CH₃ | CH₃ | | | —O—CH₂—O— | | | Psychedelic, |
| | | | | | | | | entactogen, and |
| | | | | | | | | releasing agent |
| MDMA | CH₃ | CH₃ | | | —O—CH₂—O— | | | Psychedelic, |
| | | | | | | | | entactogen, and |
| | | | | | | | | releasing agent |
| MDMC | CH₃ | CH₃ | ═O | | —O—CH₂—O— | | | Psychedelic, |
| | | | | | | | | entactogen, and |
| | | | | | | | | releasing agent |
| MMDA | | CH₃ | | | —O—CH₂—O— | | OCH3 | Stimulant, |
| | | | | | | | | psychedelic and |
| | | | | | | | | entactogen |
| MMDMA | CH₃ | CH₃ | | | —O—CH₂—O— | | OCH₃ | Psychedelic, |
| | | | | | | | | entactogen, and |
| | | | | | | | | releasing agent |
| Mescaline | | | | | OCH₃ | OCH₃ | OCH₃ | Psychedelic and |
| | | | | | | | | entactogen |
| Proscaline | | | | | OCH₃ | OCH₂CH₂CH₃ | OCH₃ | Psychedelic and |
| | | | | | | | | entactogen |
| Metaescaline | | | | | OCH₂CH₃ | OCH₃ | OCH₃ | Psychedelic and |
| | | | | | | | | entactogen |
| Allylescaline | | | | | OCH₃ | OCH₂CH₁CH₂ | OCH₃ | Psychedelic and |
| | | | | | | | | entactogen |
| Methallylescaline | | | | | OCH₃ | OCH₂C(CH₂CH₃) | OCH₃ | Psychedelic and |
| | | | | | | | | entactogen |
| Asymbescaline | | | | | OCH₂CH₃ | OCH₂CH₃ | OCH3 | Psychedelic and |
| | | | | | | | | euphoriant |
| DOM | | CH₃ | | OCH₃ | | CH₃ | OCH₃ | Psychedelic |
| DOB | | CH₃ | | OCH₃ | | Br | OCH₃ | Psychedelic |
| DOI | | CH₃ | | OCH₃ | | I | OCH₃ | Psychedelic |
| DON | | CH₃ | | OCH₃ | | NO₂ | OCH₃ | Stimulant |
| DOC | | CH₃ | | OCH₃ | | Cl | OCH₃ | Psychedelic |
| 2C-B | | | | OCH₃ | | Br | OCH₃ | Psychedelic, |
| | | | | | | | | stimulant, |
| | | | | | | | | entactogen and |
| | | | | | | | | euphoriant |
| βk-2C-B | | | ═O | OCH₃ | | Br | OCH₃ | Psychedelic, |
| | | | | | | | | stimulant, |
| | | | | | | | | entactogen and |
| | | | | | | | | euphoriant |
| 2C-C | | | | OCH₃ | | Cl | OCH₃ | Psychedelic |
| 2C-I | | | | OCH₃ | | I | OCH₃ | Psychedelic, |
| | | | | | | | | stimulant |
| 2C-D | | | | OCH₃ | | CH₃ | OCH₃ | Psychedelic, |
| | | | | | | | | stimulant |
| 2C-E | | | | OCH₃ | | CH₂—CH₃ | OCH₃ | Psychedelic |
| 2C-P | | | | OCH₃ | | CH₂—CH₃—CH₃ | OCH₃ | Entactogen, |
| | | | | | | | | euphoriant and |
| | | | | | | | | Psychedelic |
| 2C-F | | | | OCH₃ | | F | OCH₃ | Psychedelic |
| 2C-N | | | | OCH₃ | | NO₂ | OCH₃ | euphoriant |
| 2C-T-2 | | | | OCH₃ | | S—CH₂CH₃ | OCH₃ | Psychedelic |
| 2C-T-4 | | | | OCH₃ | | S—CH(CH₃)₂ | OCH₃ | Psychedelic |
| 2C-T-7 | | | | OCH₃ | | S—CH₂CH₂CH₃ | OCH₃ | Psychedelic |
| 2C-T-8 | | | | OCH₃ | | S—CH₂—C₃H5 | OCH₃ | Psychedelic |
| 2C-T-19 | | | | OCH₃ | | S—C(CH₃)₃ | OCH₃ | Psychedelic |
| 2C-T-21 | | | | OCH₃ | | S—CH₂—CH₂—F | OCH₃ | Psychedelic and |
| | | | | | | | | euphoriant |
| 25B-NBOMe | CH₂—C6H4—OCH₃ | | | OCH₃ | | Br | OCH₃ | Psychedelic |
| 25C-NBOMe | CH₂—C6H4—OCH₃ | | | OCH₃ | | Cl | OCH₃ | Psychedelic |
| 25I-NBOMe | ICH₂—C6H4—OCH₃ | | | OCH₃ | | I | OCH₃ | Psychedelic |
| 25D-NBOMe | CH₂—C6H4—OCH₃ | | | OCH₃ | | CH₂ | OCH₃ | Psychedelic |
| 25E-NBOMe | CH₂—C6H4—OCH₃ | | | OCH₃ | | CH₂—CH₃ | OCH₃ | Psychedelic |
| 25P-NBOMe | CH₂—C6H4—OCH₃ | | | OCH₃ | | CH₂—CH₃—CH₃ | OCH₃ | Psychedelic |
| 25F-NBOMe | CH₂—C6H4—OCH₃ | | | OCH₃ | | F | OCH₃ | Psychedelic |
| Mescaline- | CH₂—C6H4—OCH₃ | | | | OCH₃ | OCH₃ | OCH₃ | Psychedelic |
| NBOMe | | | | | | | | |
| 25I-NBOH | CH₂—C6H4—OH | | | OCH₃ | | I | OCH₃ | Psychedelic |
| 25C-NBOH | CH₂—C6H4—OH | | | OCH₃ | | Cl | OCH₃ | Psychedelic |
| 25B-NBOH | CH₂—C6H4—OH | | | OCH₃ | | Br | OCH₃ | Psychedelic |
| 25I-NBF | CH₂—C6H4—F | | | OCH₃ | | I | OCH₃ | Psychedelic |
| Amfepramone | C₂H5,C₂H5 | CH₃ | ═O | | | | | Anorectic |
| (diethylpropion) | | | | | | | | |

In some aspects, the substituted phenethylamine in a composition of the instant disclosure is a phenethylamine having psychoactive activity. In some aspects, the substituted phenethylamine in a composition of the instant disclosure is a phenethylamine having no known psychoactive activity.

In some aspects, compositions of the instant disclosure comprise a 2C-x phenethylamine. 2C-x (2C) phenethylamines are a family of more than 50 psychedelic substituted phenethylamines containing methoxy groups on the 2 and 5 positions of a benzene ring. A 2C compound is a compound having Formula I:

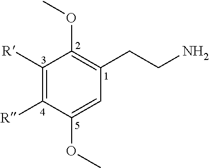

Most of these compounds also carry lipophilic substituents at the 4 position, usually resulting in more potent and more metabolically stable and longer acting compounds. Most of the currently known 2C compounds were first synthesized by Alexander Shulgin in the 1970s and 1980s and published in his book PiHKAL (Phenethylamines I Have Known And Loved). Shulgin also coined the term 2C, being an acronym for the 2 carbon atoms between the benzene ring and the amino group. Non-limiting examples of 2C compounds are shown in Table 2 below.

TABLE 2
Representative 2C compounds

| Nomenclature | R3 | R4 | 2D Structure | CAS number |
|---|---|---|---|---|
| 2C-B | H | Br | 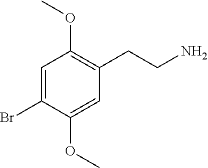 | 66142-81-2 |
| 2C-Bn | H | CH₂C₆H₅ | 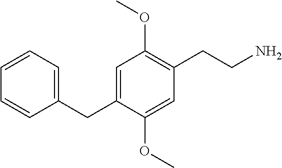 [SMILES: COc1cc(Cc2ccccc2)c(OC)cc1CCN] | |
| 2C-Bu | H | CH₂CH₂CH₂CH₃ | 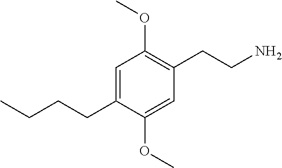 [SMILES: CCCCc1cc(OC)c(CCN)cc1OC] | |
| 2C-C | H | Cl | 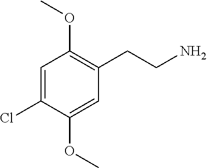 | 88441-14-9 |
| 2C-C-3 | Cl | Cl | 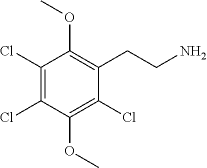 | |
| 2C-CN | H | C≡N | 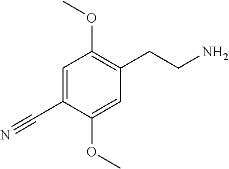 | 88441-07-0 |
| 2C-CP | H | C₃H₅ | 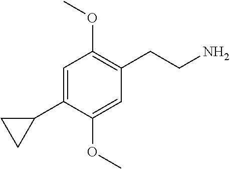 [SMILES: COc1cc(C2CC2)c(OC)cc1CCN] | |
| 2C-D | H | CH₃ | 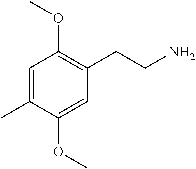 | 24333-19-5 |
| 2C-E | H | CH₂CH₃ | 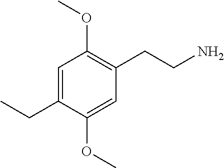 | 71539-34-9 |
| 2C-EF | H | CH₂CH₂F | 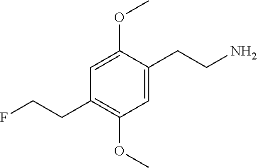 [SMILES: COc1cc(CCF)c(OC)cc1CCN] | 1222814-77-8 |
| 2C-F | H | F | 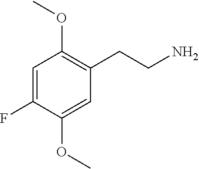 | 207740-15-6 |
| 2C-G | CH₃ | CH₃ | 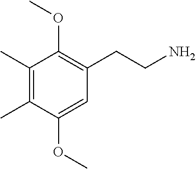 | 207740-18-9 |
| 2C-G-1 | CH₂ | | 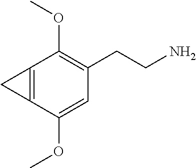 | |
| 2C-G-2 | (CH₂)₂ | | 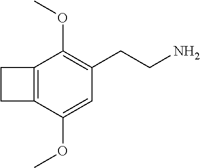 | |
| 2C-G-3 | (CH₂)₃ | | 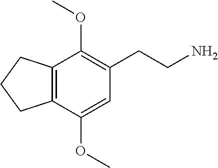 [SMILES: COc1cc(CCN)c(OC)c2c1CCC2] | 207740-19-0 |
| 2C-G-4 | (CH₂)₄ | | 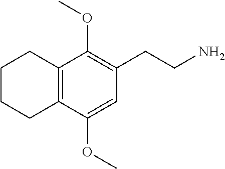 [SMILES: COc1cc(CCN)c(OC)c2c1CCCC2] | 952006-59-6 |
| 2C-G-5 | (CH₂)₅ | | 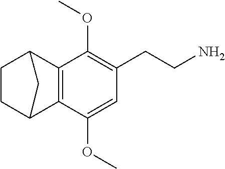 [SMILES: COc1cc(CCN)c(OC)c2c1C1CCC2C1] | 207740-20-3 |
| 2C-G-6 | (CH₂)₆ | | 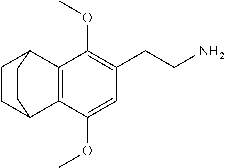 [SMILES: COc1cc(CCN)c(OC)c2c1C1CCC2CC1] | |
| 2C-G-N | (CH)₄ | | 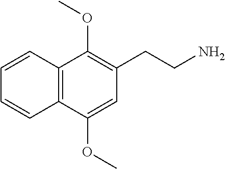 [SMILES: COc1cc(CCN)c(OC)c2ccccc12] | 207740-21-4 |
| 2C-H | H | H | 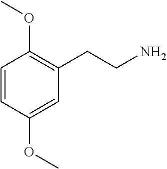 | 3600-86-0 |
| 2C-I | H | I | 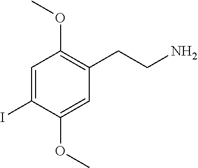 | 69587-11-7 |
| 2C-iP | H | CH(CH₃)₂ | 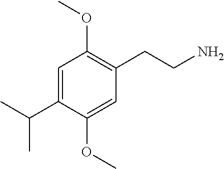 [SMILES: COc1cc(C(C)C)c(OC)cc1CCN] | 1498978-47-4 |
| 2C-N | H | NO₂ | 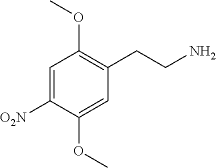 | 261789-00-8 |
| 2C-NH2 | H | NH₂ | 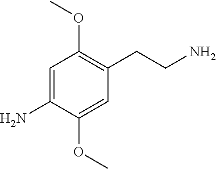 | 168699-66-9 |
| 2C-PYR | H | Pyrrolidine | 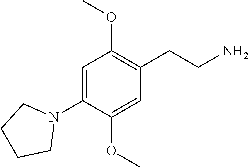 [SMILES: COc1cc(N2CCCC2)c(OC)cc1CCN] | |
| 2C-PIP | H | Piperidine | 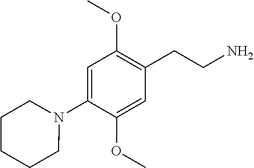 [SMILES: COc1cc(N2CCCCC2)c(OC)cc1CCN] | |
| 2C-O | H | OCH₃ | 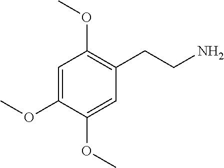 | 15394-83-9 |
| 2C-O-4 | H | OCH(CH₃)₂ | 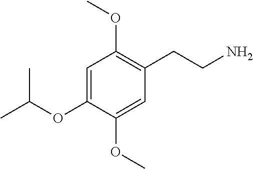 [SMILES: COc1cc(OC(C)C)c(OC)cc1CCN] | 952006-65-4 |
| 2C-MOM | H | CH₂OCH₃ | 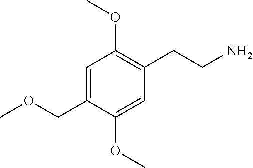 [SMILES: COCc1cc(OC)c(CCN)cc1OC] | |
| 2C-P | H | CH₂CH₂CH₃ | 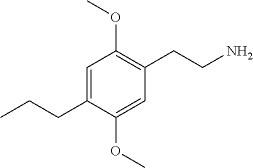 [SMILES: CCCc1cc(OC)c(CCN)cc1OC] | 207740-22-5 |
| 2C-Ph | H | C₆H₅ | 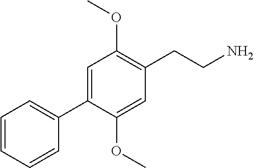 [SMILES: COc1cc(-c2ccccc2)c(OC)cc1CCN] | |
| 2C-Se | H | Se CH₃ | 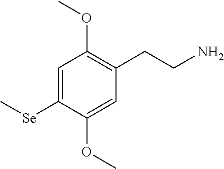 | 1189246-68-1 |
| 2C-T | H | SCH₃ | 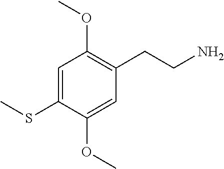 | 61638-09-3 |
| 2C-T-2 | H | SCH₂CH₃ | 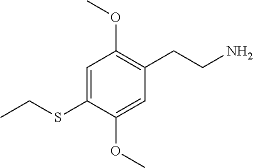 [SMILES: CCSc1cc(OC)c(CCN)cc1OC] | 207740-24-7 |
| 2C-T-3 | H | SCH₂C(═CH₂)CH₃ | 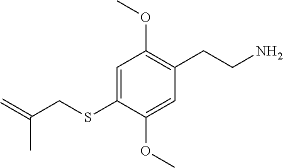 [SMILES: C=C(C)CSc1cc(OC)c(CCN)cc1OC] | 648957-40-8 |
| 2C-T-4 | H | SCH(CH₃)₂ | 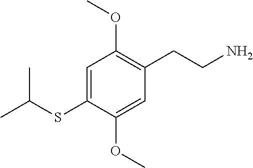 [SMILES: COc1cc(SC(C)C)c(OC)cc1CCN] | 207740-25-8 |
| 2C-T-5 | | | 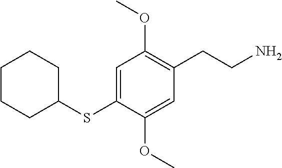 [SMILES: COc1cc(SC2CCCCC2)c(OC)cc1CCN] | |
| 2C-T-6 | | | 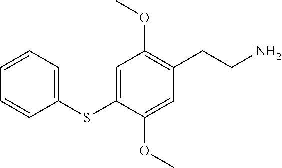 [SMILES: COc1cc(Sc2ccccc2)c(OC)cc1CCN] | |
| 2C-T-7 | H | S(CH₂)₂CH₃ | 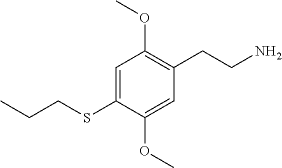 [SMILES: CCCSc1cc(OC)c(CCN)cc1OC] | 207740-26-9 |
| 2C-T-8 | H | SCH₂CH(CH₂)₂ | 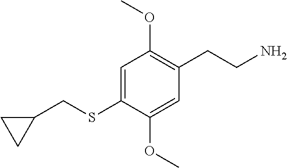 [SMILES: COc1cc(SCC2CC2)c(OC)cc1CCN] | 207740-27-0 |
| 2C-T-9 | | | 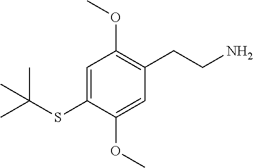 [SMILES: COc1cc(SC(C)(C)C)c(OC)cc1CCN] | 207740-28-1 |
| 2C-T-10 | | | 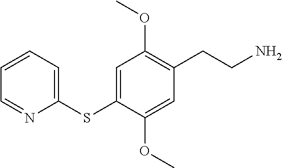 [SMILES: COc1cc(Sc2ccccn2)c(OC)cc1CCN] | |
| 2C-T-11 | | | 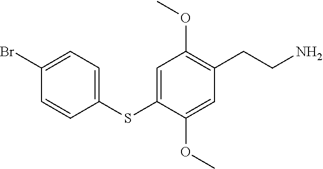 [SMILES: COc1cc(Sc2ccc(Br)cc2)c(OC)cc1CCN] | |
| 2C-T-12 | | | 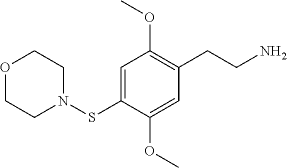 [SMILES: COc1cc(SN2CCOCC2)c(OC)cc1CCN] | |
| 2C-T-13 | H | S(CH₂)₂OCH₃ | 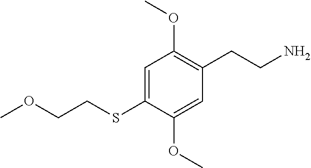 [SMILES: COCCSc1cc(OC)c(CCN)cc1OC] | 207740-30-5 |
| 2C-T-14 | | | 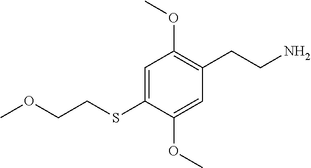 [SMILES: COCCSc1cc(OC)c(CCN)cc1OC] | |
| 2C-T-15 | H | SCH(CH₂)₂ | 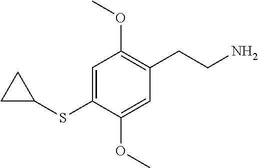 [SMILES: COc1cc(SC2CC2)c(OC)cc1CCN] | |
| 2C-T-16 | H | SCH₂CH═CH₂ | 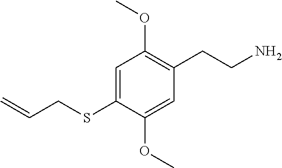 [SMILES: C=CCSc1cc(OC)c(CCN)cc1OC] | 648957-42-0 |
| 2C-T-17 | H | SCH(CH₃)CH₂CH₃ | 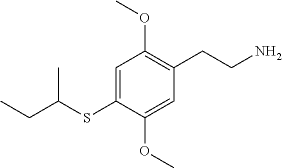 [SMILES: CCC(C)Sc1cc(OC)c(CCN)cc1OC] | 207740-32-7 |
| 2C-T-18 | | | 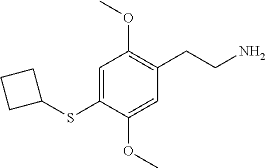 [SMILES: COc1cc(SC2CCC2)c(OC)cc1CCN] | |
| 2C-T-19 | H | SCH₂CH₂CH₂CH₃ | 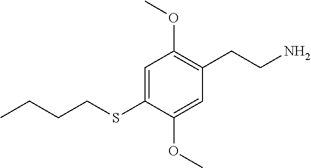 [SMILES: CCCCSc1cc(OC)c(CCN)cc1OC] | |
| 2C-T-21 | H | S(CH₂)₂F | 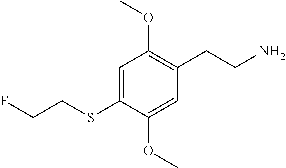 [SMILES: COc1cc(SCCF)c(OC)cc1CCN] | 207740-33-8 |
| 2C-T-21.5 | | | 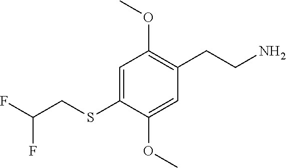 [SMILES: COc1cc(SCC(F)F)c(OC)cc1CCN] | 648957-46-4 |
| 2C-T-22 | | | 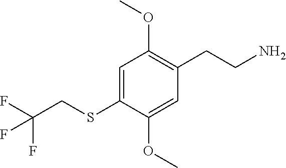 [SMILES: COc1cc(SCC(F)(F)F)c(OC)cc1CCN] | 648957-48-6 |
| 2C-T-23 | | | 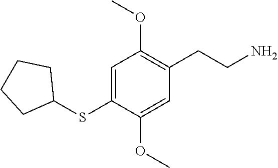 [SMILES: COc1cc(SC2CCCC2)c(OC)cc1CCN] | |
| 2C-T-24 | | | 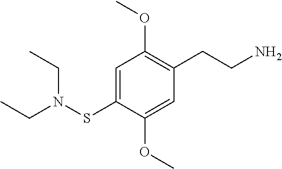 [SMILES: CCN(CC)Sc1cc(OC)c(CCN)cc1OC] | |
| 2C-T-25 | | | 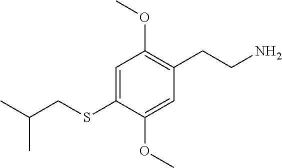 [SMILES: COc1cc(SCC(C)C)c(OC)cc1CCN] | |
| 2C-T-27 | | | 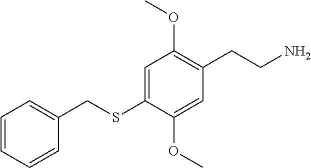 [SMILES: COc1cc(SCc2ccccc2)c(OC)cc1CCN] | 648957-52-2 |
| 2C-T-28 | | | 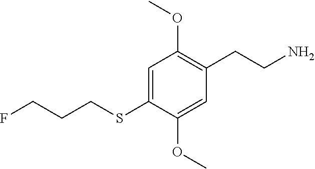 [SMILES: COc1cc(SCCCF)c(OC)cc1CCN] | 648957-54-4 |
| 2C-T-30 | | | 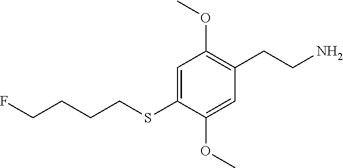 [SMILES: COc1cc(SCCCCF)c(OC)cc1CCN] | |
| 2C-T-31 | | | 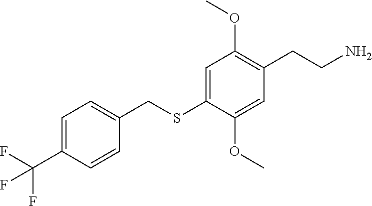 [SMILES: COc1cc(SCc2ccc(C(F)(F)F)cc2)c(OC)cc1CCN] | |
| 2C-T-32 | | | 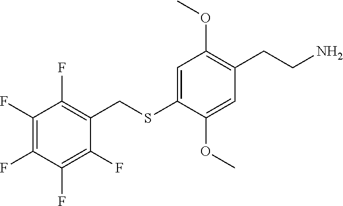 [SMILES: COc1cc(SCc2c(F)c(F)c(F)c(F)c2F)c(OC)cc1CCN] | |
| 2C-T-33 | | | 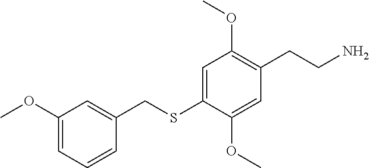 [SMILES: COc1cccc(CSc2cc(OC)c(CCN)cc2OC)c1] | |
| 2C-DFM | H | CHF₂ | 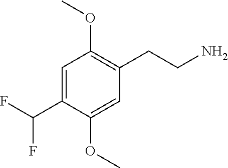 [SMILES: COc1cc(C(F)F)c(OC)cc1CCN] | |
| 2C-TFM | H | CF₃ | 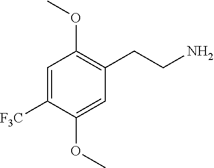 | 159277-08-4 |
| 2C-TFE | H | CH₂CF₃ | 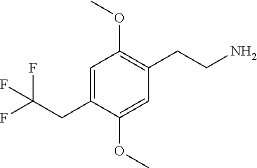 [SMILES: COc1cc(CC(F)(F)F)c(OC)cc1CCN] | |
| 2C-YN | H | C=C H | 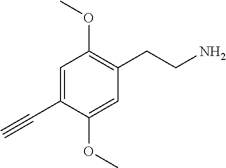 | 752982-24-4 |
| 2C-V | H | CH═CH₂ | 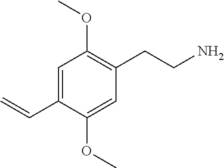 | |
| 2C-AL | H | CH₂CH═CH₂ | 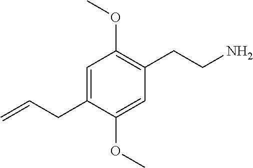 [SMILES: C=CCc1cc(OC)c(CCN)cc1OC] | |

In some aspects, the substituted phenethylamine in a composition of the instant disclosure is a 2C phenethylamine having psychoactive activity. When the substituted phenethylamine exhibits psychoactive activity, a composition of the instant disclosure can be effective at treating inflammation and psychological disorders even when administered at sub-psychoactive concentrations. In some aspects, the substituted phenethylamine in a composition of the instant disclosure is a 2C phenethylamine having no known psychoactive activity.

In some aspects, the substituted phenethylamine in a composition of the instant disclosure is a 2C compound substituted at the 4 position of the benzene ring of Formula II:

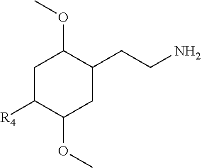

wherein R4 can be a group selected from R4 susbtituents in Table 2.

In some aspects, the substituted phenethylamine in a composition of the instant disclosure is 2C-H. The inventor surprisingly discovered that compositions comprising 2C-H can be used to treat inflammation and inflammatory disorders. A structural representation of 2C-H is shown below:

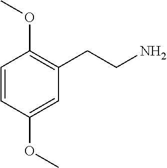

In some aspects, the composition comprises 2C-H in a unit dose comprising 2C-H at a concentration ranging from about 1 mg to about 100 mg, at a concentration ranging from about 100 mg to about 200 mg, at a concentration of about 25 mg, at a concentration of about 50 mg, at a concentration of about 100 mg, at a concentration of about 150 mg, or at a concentration of about 200 mg.

In some aspects, the substituted phenethylamine in a composition of the instant disclosure is 2-(4-lodo-2,5-dimethoxyphenyl)ethan-1-amine (2C-I). The structural representation of 2C-I is shown below:

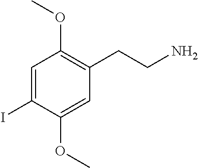

2C-I is a psychoactive compound that can cause psychedelic activity at a threshold unit dose of about 2 mg. Recreationally, a light dose of 2C-I can range from about 5 mg to about 10 mg, a common dose of 2C-I can range from about 10 mg to about 20 mg, a strong dose of 2C-I can range from about 20 mg to about 30 mg, and a heavy dose of 2C-I is about 30 mg and above. As explained above, when a substituted phenethylamine is a compound that exhibits psychoactive activity, a composition of the instant disclosure can be effective at treating inflammation and psychological disorders even when administered at sub-psychoactive doses or concentrations. In some aspects, the composition comprises 2C-I in a unit dose comprising 2C-I at a concentration of about 2 mg or below. It will be appreciated that a psychoactive dose can and will vary depending on the subject, the frequency of administration of the compound, the weight and height of the subject, formulations of the active compound, and the route of administration among other factors. For instance, if a substituted phenethylamine of the instant disclosure is administered intravenously, a unit dose of the compound can be any dose that can maintain the neurological activity of the compound but that does not induce psychoactive activity in the subject. In such aspects, unit doses of a compound are significantly higher than the threshold dose when taken orally or nasally during recreational use. Accordingly, a sub-psychoactive but neurologically effective dose of a compound of the instant disclosure will also vary depending on the subject, the weight and height of the subject, the frequency of administration of the compound, formulations of the active compound, and the route of administration among other factors.

In some aspects, the substituted phenethylamine in a composition of the instant disclosure is 4-Bromo-2,5-dimethoxyphenethylamine (2C-B). The structural representation of 2C-B is shown below:

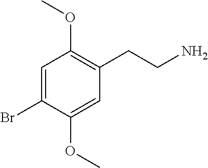

2C-B is a psychoactive compound that can cause psychedelic activity at a threshold unit dose of about 5 mg. Recreationally, a light dose of 2C-B can range from about 10 mg to about 15 mg, a common dose of 2C-B can range from about 15 mg to about 25 mg, a strong dose of 2C-B can range from about 25 mg to about 45 mg, and a heavy dose of 2C-B is about 45 mg and above. As explained above, a sub-psychoactive but neurologically effective dose of a compound of the instant disclosure will vary depending on the subject, the weight and height of the subject, the frequency of administration of the compound, formulations of the active compound, and the route of administration among other factors. In some aspects, the composition comprises 2C-B in a unit dose comprising 2C-B at a concentration of about 5 mg or below.

As explained herein above, a sub-psychoactive but neurologically effective dose of a compound of the instant disclosure will vary depending on the subject, the weight and height of the subject, the frequency of administration of the compound, formulations of the active compound, and the route of administration among other factors, and can be significantly higher or lower than the threshold dose of the compound when taken recreationally. In some aspects, the composition comprises 2C-B in a unit dose comprising 2C-B at a concentration ranging from about 1 mg to about 10 mg, at a concentration ranging from about 1 mg to about 20 mg, at a concentration ranging from about 1 mg to about 100 mg, at a concentration of about 10 mg, at a concentration of about 15 mg, at a concentration of about 20 mg, or at a concentration of about 25 mg.

In some aspects, the substituted phenethylamine in a composition of the instant disclosure is 4-Ethyl-2,5-dimethoxyphenethylamine (2C-E). The structural representation of 2C-E is shown below:

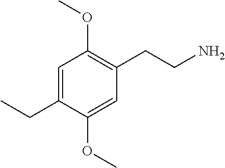

2C-E is a psychoactive compound that can cause psychedelic activity at a threshold unit dose of about 2 mg. Recreationally, a light dose of 2C-E can range from about 5 mg to about 10 mg, a common dose of 2C-E can range from about 10 mg to about 15 mg, a strong dose of 2C-B can range from about 15 mg to about 30 mg, and a heavy dose of 2C-E is about 30 mg and above. As explained above, a sub-psychoactive but neurologically effective dose of a compound of the instant disclosure will vary depending on the subject, the weight and height of the subject, the frequency of administration of the compound, formulations of the active compound, and the route of administration among other factors. In some aspects, the composition comprises 2C-E in a unit dose comprising 2C-E at a concentration of about 2 mg or below. However, a sub-psychoactive but neurologically effective dose of a compound of the instant disclosure will vary depending on the subject, the weight and height of the subject, the frequency of administration of the compound, formulations of the active compound, and the route of administration among other factors, and can be significantly higher or lower than the threshold dose of the compound when taken recreationally.

### (b) Compositions

Compositions of the instant disclosure can be formulated in dosage unit formulations containing conventional nontoxic pharmaceutically acceptable adjuvants, excipients, and vehicles as desired. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

A composition of the instant disclosure can be formulated and administered to a subject by several different means. For instance, a composition may generally be administered parenterally, intraperitoneally, intravascularly, trans-dermally, subcutaneously, rectally, or intrapulmonarily in dosage unit formulations containing conventional nontoxic pharmaceutically acceptable adjuvants, carriers, excipients, and vehicles as desired.

In those aspects administered orally, compositions may be administered in any orally acceptable dosage form including, but not limited to a tablet, including a suspension tablet, a chewable tablet, an effervescent tablet or caplet; a pill; a powder such as a sterile packaged powder, a dispensable powder, or an effervescent powder; a capsule including both soft or hard gelatin capsules such as HPMC capsules; a lozenge; a sachet; a sprinkle; a reconstitutable powder or shake; a troche; pellets; granules; liquids; suspensions; emulsions; or semisolids and gels. Alternatively, the pharmaceutical compositions may be incorporated into a food product or powder for mixing with a liquid or administered orally after only mixing with a non-foodstuff liquid. Capsule and tablet formulations may include, but are not limited to binders, lubricants, and diluents. Aqueous suspension formulations may include but are not limited to dispersants, flavor-modifying agents, taste-masking agents, and coloring agents.

Compositions provided herein may also be liquid formulations including, but not limited to, aqueous or oily suspensions, solutions, emulsions, syrups, and elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethyl cellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

Compositions provided herein may also be formulated as suppositories. In these embodiments, the composition may include a suitable non-irritating excipient that is solid at room temperature but liquid at rectal temperature and therefore will melt in the rectum to release the drug. Non-limiting examples of suitable excipients for rectal suppository embodiments include cocoa butter, beeswax, and polyethylene glycols.

Compositions provided herein may also be formulated for inhalation, which may be in a form including, but not limited to, a solution, suspension, or emulsion that may be administered as a dry powder or in the form of an aerosol using a propellant, such as dichlorodifluoromethane or trichlorofluoromethane.

Compositions provided herein may also be formulated as transdermal formulations, for example as a suitable ointment, lotion, cream, pastes, medicated plaster, patch, or membrane that includes but is not limited to a compound, according to this disclosure, suspended or dissolved in one or more carriers. Non-limiting examples of suitable carriers for transdermal embodiments include mineral oil, liquid petrolatum, white petrolatum, propylene glycol, polyoxyethylene, polyoxypropylene compound, emulsifying wax, sorbitan monostearate, Polysorbate 60, cetyl esters wax, cetearyl alcohol, 2-octyldodecanol, benzyl alcohol and water. For these embodiments, the molecular weight of the composition may range from about 1 to about 50 Daltons.

Compositions provided herein may also be formulated for parenteral administration including, but not limited to, intravenously, by injection, or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

Compositions provided herein may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

### A. Excipients and Carriers

Formulations of various aspects may include the composition of the disclosure, along with an excipient and/or pharmaceutically acceptable carrier. Non-limiting examples of excipients include preservatives (antioxidants), flavor-modifying agents, coloring agents, chelating agents, antimicrobial agents, stabilizers, surfactants, tonicity agents such as NaCl, suspending agents, release-controlling polymers, and any combination thereof. As used herein, the term “pharmaceutically acceptable carrier” is intended to include any and all solvents, dispersion media, coatings, antibacterial and antifungal agents, isotonic and absorption delaying agents, and other excipients compatible with pharmaceutical administration. The use of such media and agents for pharmaceutically active substances is well known in the art. It will be recognized that carriers can provide more than one function in a composition. A more detailed description of carriers can be as described below.

i. Preservatives

Non-limiting examples of preservatives include, but are not limited to, ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-caraotene, beta-apocarotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate), flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanillic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivatives, vitamin Q10, wheat germ oil, zeaxanthin, or combinations thereof.

ii. Chelating Agents

A chelating agent may be included as an excipient to immobilize oxidative groups, including but not limited to metal ions, in order to inhibit the oxidative degradation of the morphinan by these oxidative groups. Non-limiting examples of chelating agents include lysine, methionine, glycine, gluconate, polysaccharides, glutamate, aspartate, and disodium ethylenediaminetetraacetate (Na2EDTA).

iii. Flavor-Modifying Agents

Suitable flavor-modifying agents include flavorants, taste-masking agents, sweeteners, and the like. Flavorants include, but are not limited to, synthetic flavor oils and flavoring aromatics and/or natural oils, extracts from plants, leaves, flowers, fruits, and combinations thereof. Other non-limiting examples of flavors include cinnamon oils, oil of wintergreen, peppermint oils, clover oil, hay oil, anise oil, eucalyptus, vanilla, citrus oils such as lemon oil, orange oil, grape and grapefruit oil, fruit essences including apple, peach, pear, strawberry, raspberry, cherry, plum, pineapple, and apricot.

Taste-masking agents include, but are not limited to, cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other aspects, additional taste-masking agents contemplated are those described in U.S. Pat. Nos. 4,851,226; 5,075,114; and 5,876,759, each of which is hereby incorporated by reference in its entirety.

Non-limiting examples of sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, sylitol, hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

iv. Colorants

Depending upon the aspect, it may be desirable to include a coloring agent. Suitable color additives include, but are not limited to, food, drug, and cosmetic colors (FD&C), drug and cosmetic colors (D&C), or external drug and cosmetic colors (Ext. D&C). These colors or dyes, along with their corresponding lakes, and certain natural and derived colorants, may be suitable for use in various aspects.

v. Chelating Agents

A chelating agent may be included as an excipient to immobilize oxidative groups, including but not limited to metal ions, in order to inhibit the oxidative degradation of the morphinan by these oxidative groups. Non-limiting examples of chelating agents include lysine, methionine, glycine, gluconate, polysaccharides, glutamate, aspartate, and disodium ethylenediaminetetraacetate (Na2EDTA).

vi. Antimicrobial Agents

An antimicrobial agent may be included as an excipient to minimize the degradation of the compound according to this disclosure by microbial agents, including but not limited to, bacteria and fungi. Non-limiting examples of antimicrobials include parabens, chlorobutanol, phenol, calcium propionate, sodium nitrate, Na₂EDTA, and sulfites, including but not limited to sulfur dioxide, sodium bisulfite, and potassium hydrogen sulfite.

vii. Stabilizers

In some aspects, isotonifiers, sometimes known as “stabilizers”, are added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive, which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers such as polyvinylpyrrolidone monosaccharides, such as xylose, mannose, fructose, glucose; disaccharides such as lactose, maltose, sucrose and trisaccacharides such as raffinose; and polysaccharides such as dextran. In some aspects, the composition does not include stabilizers.

viii. Antimicrobial Agents

An antimicrobial agent may be included as an excipient to minimize the degradation of the compound according to this disclosure by microbial agents, including but not limited to, bacteria and fungi. Non-limiting examples of antimicrobials include para-bens, chlorobutanol, phenol, calcium propionate, sodium nitrate, Na₂EDTA, and sulfites, including but not limited to sulfur dioxide, sodium bisulfite, and potassium hydrogen sulfite.

ix. Stabilizers

In some aspects, isotonifiers, sometimes known as “stabilizers”, are added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive, which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers such as polyvinylpyrrolidone monosaccharides, such as xylose, mannose, fructose, glucose; disaccharides such as lactose, maltose, sucrose and trisaccacharides such as raffinose; and polysaccharides such as dextran.

x. Surfactants

Non-ionic surfactants or detergents (also known as “wetting agents”) can be used to help solubilize the therapeutic agent, as well as to protect the active ingredient against agitation-induced aggregation. Suitable non-ionic surfactants include polysorbates (20, 80, etc.), polyoxamers (184, 188, etc.), Pluronic polyols, and polyoxyethylene sorbitan monoethers (TWEEN®-20, TWEEN®-80, etc.).

### B. Encapsulation

A composition of the instant disclosure can comprise a substituted phenethylamine encapsulated in a drug vehicle. As used herein, the term “encapsulated” refers to an active ingredient such as a substituted phenethylamine of the instant disclosure encapsulated in a drug carrier or drug vehicle. A wide variety of drug delivery systems have been developed and studied, each of which has unique advantages and disadvantages. Non-limiting examples of drug vehicles include liposomes, polymeric micelles, microspheres, and nanoparticles among others. Different methods of attaching the drug to the carrier have been implemented, including adsorption, integration into the bulk structure, encapsulation, and covalent bonding. Different types of drug vehicles utilize different methods of attachment, and some vehicles can even implement a variety of attachment methods.

Non-limiting examples of nanoparticles include nano diamonds, nanofibers, protein-DNA complexes, protein-drug complexes, protein-drug conjugates, erythrocytes, virosomes, and dendrimers. Encapsulating drug compounds such as substituted phenethylamines of the instant disclosure can provide numerous benefits such as increasing the stability and life of the compound being encapsulated, facilitate the manipulation of the product, and provide for the controlled release of the contents.

Polymeric micelles are drug vehicles formed by the aggregation of some amphiphilic molecules with an amphiphilic block copolymer. These vehicles form at some high concentration specific to the compounds used, called the critical micelle concentration. The addition of an amphiphilic block copolymer effectively lowers this critical micelle concentration by shifting the monomer exchange equilibrium.

Microspheres are hollow, micron-sized vehicles often formed via self-assembly of polymeric compounds which are most often used to encapsulate the active drug for delivery. Drug release from microspheres is often achieved by diffusion through pores in the microsphere structure or by degradation of the microsphere shell. Some assembly techniques, such as precision particle fabrication (PPF), can create microspheres capable of sustained control over drug release.

Liposomes are artificially spherical vesicles prepared from naturally derived phospholipid. They entail one or more lipid bilayers with discrete aqueous spaces. They are well established for a range of pharmaceutical and biomedical applications with the unique capability of entrapment of both hydrophilic (polar) and hydrophobic (nonpolar) compounds due to their amphipathic nature in aqueous media. For instance, hydrophobic compounds entrap in the bilayer membrane, while hydrophilic compounds encapsulate in the aqueous core. Liposomes serve as DDSs due to their versatile structure; biocompatibility; and the fact they are naturally nontoxic, non-immunogenic, and biodegradable. Liposomes have several advantages contributing to drug delivery. They have a role enhancing drug solubility, serving as a sustained release system, providing targeted drug delivery, reducing the toxic effect of drugs, providing protection against drug degradation, enhancing circulation half-life of APIs, being effective in overcoming multidrug resistance, improving the therapeutic index of the entrapped drug, and protecting APIs against their surrounding environment.

Numerous factors define liposomes properties such as the lipid composition, number of lipid bilayers, size, surface charge, and the method of preparation. Liposomal vesicles vary in size between 0.025 μm to 2.5 μm. They can be categorised according to the number of their layers (also referred to as lamellae): unilamellar (consisting of single phospholipid bilayer) or multilamellar (consisting of more than one unilamellar separated by layers of water (>500 nm)). Unilamellar vesicles are subdivided into small unilamellar vesicles (20-100 nm) and large unilamellar vesicles (>100 nm). Both the size and the number of lamellae in the liposomal structure are considered to be the most crucial factors affecting the vesicles half-life and the quantity of API that is to be encapsulated. This unique and flexible variety in the liposomal structure distinguishes liposomes as the preferred carriers for a broad spectrum of therapeutic agents.

Physical and chemical stability of the liposomes in terms of size distribution, entrapment efficiency, and minimal degradation of liposomal apparatuses is the major limiting step for drug delivery using this system. Chemical degradation of liposomes mainly occurs at the phospholipid bilayers level, in which two different reactions might develop: (i) hydrolysis of the ester bonds between fatty acids and glycerol backbone, and (ii) peroxidation of any available unsaturated acyl chain. These two reactions might lead to the development of short-chain lipids; subsequently, soluble derivatives will appear in the membrane that would significantly reduce the quality and stability of the liposomal system. With respect to physical instability, liposomes might undergo aggregation/flocculation and fusion/coalescence, which can ultimately change vesicle size and lead to significant loss of the encapsulated API.

Several factors that have an influence on liposomal system stability, such as liposomal composition (e.g., phospholipids-lipids with high phase transition temperatures), fatty acid side-chains, polar head chemistry, chain length, and the degree of unsaturation, can maintain liposomal rigidity and phospholipid:cholesterol molar ratio (crucial for the liposomal stability and controlling drug release).

Phospholipids are the main building blocks of liposomes. These biomolecules are also the main components building the biological membranes. They are amphiphilic molecules that consist of a polar head (water soluble hydroxy groups) and insoluble backbone. Liposomes can be zwitterionic, positively or negatively charged, or uncharged. This is dependent on the polar head charge. There are two types of lipids currently utilized for liposome preparation: naturally occurring or synthesized double-chain lipids (consisting of phosphorus polar head and glycerol backbone) and sterols (e.g., cholesterol).

The most known lipids used in the liposomal formulations are phosphatidylcholine (zwitterionic), phosphatidylglycerol (negatively charged), phosphatidic acid, phosphatidylethanolamine (zwitterionic), and phosphatidylserine (negatively charged). Positively charged lipids (e.g., N-[1-(2,3-dioleyloxy)propyl]-N,N,N-triethylammonium (DOTMA) and 1,2-dioleoyl-3-trimethylammoniopropane (DOTAP)) are mainly used for gene delivery, as they interact with the negatively charged deoxyribonucleic acid (DNA) and negatively charged APIs.

Cholesterol is another strategic component of liposomes. It has a modulatory effect on the properties of the lipid bilayer of the liposomes. It can control the stoutness in the liposome structure and increase the packing between the phospholipid molecules, resulting in more ordered conformation in the aliphatic tail region, reduced micropolarity, reduced bilayer flexibility to the surrounding molecules (especially water-soluble molecules), and increases in the microviscosity of the bilayer. Cholesterol is also crucial for structural stability of liposomal membranes against intestinal environmental stress. Cholesterol was found to influence liposomes size (increasing cholesterol concentration increases liposomes size in addition to shape transition), provide permeability and fluidity, and consequently modulate the release of hydrophilic compounds from liposomes.

The lipid bilayer of a liposome may fuse with other bilayers (e.g., the cell membrane), thus delivering the contents of the liposome to cells. Phospholipids generally comprise two fatty acids linked through glycerol phosphate to one of a variety of polar groups. Non-limiting examples of phospholids suitable for liposomes include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE), or any combination thereof.

The fatty acid chains may range from about 6 to about 26 carbon atoms in length, and the lipid chains may be saturated or unsaturated. Non-limiting examples of suitable fatty acid chains include (common name presented in parentheses) n-dodecanoate (laurate), n-tetradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all-cis-9,12,15-octadecatrienoate (linolenate), and all-cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and any combination thereof.

The phospholipids can come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-diheptyloxacarbocyanine iodide, 1,1′-didodecyl-3,3,3′,3′-tetramethylindocarbocyanine perchloarate, 1,1′-dioleyl-3,3,3′,3′-tetramethylindo carbocyanine methanesulfonate, N,4-(dilinoleylaminostyryl)-N-methylpyridinium iodide, or 1,1-dilinoleyl-3,3,3′,3′-tetramethylindocarbocyanine perchloarate.

Liposomes can optionally comprise sphingolipids, in which spingosine is the structural counterpart of glycerol and one of the one fatty acids of a phosphoglyceride, or cholesterol, a major component of animal cell membranes. Liposomes may optionally, contain pegylated lipids, which are lipids covalently linked to polymers of polyethylene glycol (PEG). The PEGylated lipids may generally increase the amount of compound that can be incorporated into the liposomes. PEGs may range in size from about 500 to about 10,000 Daltons. A suitable PEGylated phospholipid is dipalmitoyl PE bearing PEG 5,000.

Liposomes can further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the substituted phenethylamines of the instant disclosure can be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, and Verrico et al., PAIN, 2020 Sep. 1; 161(9): 2191-2202, the disclosures of which are hereby incorporated by reference in their entirety. For example, liposomes can be prepared by sonicating lipids in an aqueous solution, solvent injection, lipid hydration, reverse evaporation, or freeze drying by repeated freezing and thawing. The liposomes can be multilamellar, which have many layers like an onion, or unilamellar.

As would be apparent to one of ordinary skill, all the parameters that govern liposome formation may be varied. These parameters include, but are not limited to, temperature, pH, concentration of active pharmaceutical ingredient, concentration and composition of lipid, concentration of multivalent cations, rate of mixing, presence of and concentration of solvent.

### II. Method of Treating

Another aspect of the present disclosure encompasses a method of treating inflammation and inflammatory disorder or psychological disorder in a subject in need thereof. The method comprises administering to the subject a therapeutically effective amount of a composition comprising a substituted phenethylamine. Importantly, using the compositions of the instant disclosure, methods of the instant disclosure can treat inflammation and psychological disorders even when administered at sub-hallucinogenic doses. Compositions comprising a substituted phenethylamine can be as described in Section I.

A composition of the instant disclosure can be administered to a subject by several different means. For instance, a composition may generally be administered parenterally, intraperitoneally, intravascularly, transdermally, subcutaneously, rectally, or intrapulmonarily in dosage unit formulations containing conventional nontoxic pharmaceutically acceptable adjuvants, carriers, excipients, and vehicles as desired.

The subject can be a human, a livestock animal, a companion animal, a lab animal, or a zoological animal. In one aspect, the subject may be a rodent, e.g., a mouse, a rat, a guinea pig, etc. Non-limiting examples of suitable livestock animals may include pigs, cows, horses, goats, sheep, llamas, and alpacas. Non-limiting examples of companion animals may include pets such as dogs, cats, rabbits, and birds. As used herein, a “zoological animal” refers to an animal that may be found in a zoo. Such animals may include non-human primates, large cats, wolves, and bears. Non-limiting examples of a laboratory animal may include rodents, canines, felines, and non-human primates. Non-limiting examples of rodents may include mice, rats, guinea pigs, etc.

In some aspects, the subject is a human subject. In some aspects, the disorder is coronary artery disease (narrowing of the blood vessels that supply blood to the heart).

### (a) Inflammation

One aspect of the instant disclosure encompasses a method of treating inflammation. The method comprises administering a therapeutically effective amount of a substituted phenethylamine to a subject in need thereof.

In some aspects, the method comprises administering 2C-H. In some aspects, the method comprises administering 2C-H at therapeutically effective amounts ranging from about 0.1 mg/kg to about 1 mg/kg body weight of the subject. In some aspects, the method comprises administering 2C-H in a unit dose comprising 2C-H at a concentration ranging from about 1 mg to about 100 mg, at a concentration ranging from about 100 mg to about 200 mg, at a concentration of about 25 mg, at a concentration of about 50 mg, at a concentration of about 100 mg, at a concentration of about 150 mg, or at a concentration of about 200 mg.

Inflammation is part of the complex biological response of body tissues to harmful stimuli, such as pathogens, damaged cells, or irritants, and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair.

Inflammation is not a synonym for infection. Infection describes the interaction between the action of microbial invasion and the reaction of the body's inflammatory response—the two components are considered together when discussing an infection, and the word is used to imply a microbial invasive cause for the observed inflammatory reaction. Inflammation, on the other hand, describes purely the body's immunovascular response—whatever the cause may be.

Inflammation can be classified as either acute or chronic. Acute inflammation is the initial response of the body to harmful stimuli and is achieved by the increased movement of plasma and leukocytes (in particular granulocytes) from the blood into the injured tissues. A series of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation, such as mononuclear cells, and is characterized by simultaneous destruction and healing of the tissue from the inflammatory process.

### A. Inflammatory Disorders

Non-limiting examples of disorders associated with, caused by, or resulting from inflammation include Inflammatory disorders include encephalitis, myelitis, meningitis; arachnoiditis; neuritis; dacryoadenitis; scleritis; episcleritis; keratitis; retinitis; chorioretinitis; blepharitis; conjunctivitis; uveitis; otitis externa; otitis media; labyrinthitis; mastoiditis; carditis; endocarditis; myocarditis; pericarditis; vasculitis; arteritis; phlebitis; capillaritis; sinusitis; rhinitis; pharyngitis; laryngitis; tracheitis; bronchitis; bronchiolitis; pneumonitis; pleuritis; mediastinitis; stomatitis; gingivitis; gingivostomatitis; glossitis; tonsillitis; sialadenitis/parotitis; cheilitis; pulpitis; gnathitis; esophagitis; gastritis; gastroenteritis; enteritis; colitis; enterocolitis; duodenitis; ileitis; caecitis; appendicitis; proctitis; hepatitis; ascending cholangitis; cholecystitis; pancreatitis; peritonitis; dermatitis; folliculitis; cellulitis; hidradenitis; arthritis; dermatomyositis; myositis; synovitis/tenosynovitis; bursitis; enthesitis; fasciitis; capsulitis; epicondylitis; tendinitis; panniculitis; osteochondritis: osteitis/osteomyelitis; spondylitis; periostitis; chondritis; nephritis; glomerulonephritis; pyelonephritis; ureteritis; cystitis; urethritis; oophoritis; salpingitis; endometritis; parametritis; cervicitis; vaginitis; vulvitis; mastitis; orchitis; epididymitis; prostatitis; seminal vesiculitis; balanitis; posthitis; balanoposthitis; chorioamnionitis; funisitis; omphalitis; insulitis; hypophysitis; thyroiditis; parathyroiditis; adrenalitis; lymphangitis; lymphadenitis

### B. Acute Inflammation

In some aspects, a method of the instant disclosure comprises treating acute inflammation. Acute inflammation occurs immediately upon injury, lasting only a few days. Cytokines and chemokines promote the migration of neutrophils and macrophages to the site of inflammation. Pathogens, allergens, toxins, burns, and frostbite are some of the typical causes of acute inflammation. Acute inflammation can be a defensive mechanism to protect tissues against injury. Inflammation lasting 2-6 weeks is designated subacute inflammation. Acute inflammation may be regarded as the first line of defense against injury. Acute inflammatory response requires constant stimulation to be sustained. Inflammatory mediators are short-lived and are quickly degraded in the tissue. Hence, acute inflammation begins to cease once the stimulus has been removed. Acute inflammation can be categorized as mild, moderate, severe, and systemic severe inflammation. Symptoms of mild, moderate, severe, and systemic severe inflammation can and will vary depending on the type of inflammation and can be as recognized in the art for each type of inflammation. For instance, a mild and localized bacterial infection on the skin can develop into a systemic severe inflammation during sepsis.

Acute inflammation is a short-term process, usually appearing within a few minutes or hours and begins to cease upon the removal of the injurious stimulus. It involves a coordinated and systemic mobilization response locally of various immune, endocrine, and neurological mediators of acute inflammation. In a normal healthy response, it becomes activated, clears the pathogen or source of injury, begins a repair process, and then ceases. It is characterized by five cardinal signs: pain, calor heat, redness, swelling, and loss of function. Redness and heat are due to increased blood flow at body core temperature to the inflamed site; swelling is caused by accumulation of fluid; and pain is due to the release of chemicals such as bradykinin and histamine that stimulate nerve endings. Loss of function has multiple causes.

However, an infectious organism can escape the confines of the immediate tissue via the circulatory system or lymphatic system, where it may spread to other parts of the body to cause severe acute inflammation. If an organism is not contained by the actions of acute inflammation, it may gain access to the lymphatic system via nearby lymph vessels. An infection of the lymph vessels is known as lymphangitis, and infection of a lymph node is known as lymphadenitis. When lymph nodes cannot destroy all pathogens, the infection spreads further. A pathogen can gain access to the bloodstream through lymphatic drainage into the circulatory system. When inflammation overwhelms the host, systemic inflammatory response syndrome is diagnosed. When it is due to infection, the term sepsis is applied, with the term's bacteremia being applied specifically for bacterial sepsis and viremia specifically to viral sepsis as is the case with COVID-19. Vasodilation and organ dysfunction are serious problems associated with widespread infection that may lead to septic shock and death.

In some aspects, the method comprises administering a therapeutically effective amount of a composition comprising a substituted phenethylamine. In some aspects, the substituted phenethylamine is a 2C compound. In some aspects, the method comprises administering a 2C compound of Formula II.

In some aspects, the method comprises administering 2C-H. In some aspects, the subject is experiencing acute inflammation and 2C-H is administered in a unit dose comprising 2C-H at a concentration ranging from about 1 mg to about 100 mg, at a concentration ranging from about 100 mg to about 200 mg, at a concentration of about 25 mg, at a concentration of about 50 mg, at a concentration of about 100 mg, at a concentration of about 150 mg, or at a concentration of about 200 mg. In some aspects, 2C-H is administered at a therapeutically effective amount ranging from about 0.1 mg/kg to about 1 mg/kg body weight of the subject.

In some aspects, the subject is experiencing mild acute inflammation and 2C-H is administered in a unit dose comprising 2C-H at a concentration of about 25 mg. In some aspects, the subject is experiencing mild acute inflammation and 2C-H is administered in a unit dose comprising 2C-H at a concentration of about 25 mg. In some aspects, the subject is experiencing moderate acute inflammation and 2C-H is administered in a unit dose comprising 2C-H at a concentration of about 50 mg. In some aspects, the subject is experiencing severe acute inflammation and 2C-H is administered in a unit dose comprising 2C-H at a concentration of about 100 mg. In some aspects, the subject is experiencing systemic acute inflammation and 2C-H is administered in a unit dose comprising 2C-H at a concentration of about 150 mg. In some aspects, the subject is experiencing systemic acute inflammation and 2C-H is administered in a unit dose comprising 2C-H at a concentration of about 200 mg.

### C. Chronic Inflammation

inflammatory arthritis, systemic lupus, sarcoidosis, and asthma

In some aspects, a method of the instant disclosure comprises treating chronic inflammation. Chronic systemic inflammation (SI) is the result of release of pro-inflammatory cytokines from immune-related cells and the chronic activation of the innate immune system. Chronic inflammation can last for months or years. Macrophages, lymphocytes, and plasma cells predominate in chronic inflammation, in contrast to the neutrophils that predominate in acute inflammation. Chronic systemic inflammation can contribute to the development or progression of certain conditions such as cardiovascular disease, cancer, diabetes mellitus, chronic kidney disease, non-alcoholic fatty liver disease, allergies, autoimmune and neurodegenerative disorders, coronary heart disease, and chronic obstructive pulmonary disease (COPD) are examples of diseases mediated by chronic inflammation. Obesity, smoking, stress and insufficient diet are some of the factors that promote chronic inflammation. Common signs and symptoms that develop during chronic inflammation include body pain, arthralgia, myalgia, chronic fatigue and insomnia, depression, anxiety, and mood disorders, gastrointestinal complications such as constipation, diarrhea, and acid reflux, weight gain or loss, and frequent infections.

In some aspects, the inflammation is chronic inflammation and the composition is administered in a unit dose comprising 2C-H at a concentration ranging from about 1 mg to about 100 mg, at a concentration of about 25 mg, at a concentration of about 50 mg, or at a concentration of about 100 mg. In some aspects, 2C-H is administered at a therapeutically effective amount ranging from about 0.1 mg/kg to about 1 mg/kg body weight of the subject.

In some aspects, the subject is experiencing mild chronic inflammation and the composition is administered in a unit dose comprising 2C-H at a concentration of about 25 mg. In some aspects, the subject is experiencing moderate chronic inflammation and wherein the composition is administered in a unit dose comprising 2C-H at a concentration of about 50 mg. In some aspects, the subject is experiencing severe chronic inflammation and wherein the composition is administered in a unit dose comprising 2C-H at a concentration of about 100 mg.

### (b) Psychological Disorders

One aspect of the instant disclosure encompasses a method of treating a psychological disorder. The method comprises administering a therapeutically effective amount of a substituted phenethylamine to a subject in need thereof.

In some aspects, the method comprises administering 2C-B. In some aspects, the method comprises administering 2C-B at a therapeutic amounts ranging from about 0.1 mg/kg to about 1 mg/kg body weight of the subject. In some aspects, the method comprises administering 2C-B in a unit dose comprising 2C-B at a concentration ranging from about 1 mg to about 10 mg, from about 1 mg to about 20 mg, from about 1 mg to about 100 mg, at about 10 mg, at about 15 mg, at about 20 mg, or at about 25 mg.

The term psychological disorders is sometimes used to refer to what are more frequently known as mental disorders or psychiatric disorders. Mental disorders are patterns of behavioral or psychological symptoms that impact multiple areas of life. These disorders create distress for the person experiencing the symptoms. Non-limiting examples of psychological disorders include anxiety disorder, bipolar disorder, dementia, ADHD, schizophrenia, OCD, Autism, PTSD, addiction/substance abuse, various forms of depression, post-traumatic stress disorder, more commonly known as PTSD, schizophrenia, and suicidal thoughts.

### (c) Administration

A composition of the instant disclosure can be administered to a subject by several different means. For instance, a composition can generally be administered parenterally, buccally, nasally, by inhalation, intraperitoneally, intravascularly, transdermally, subcutaneously, rectally, or intrapulmonarily. The term parenteral as used herein includes subcutaneous, intravenous, intramuscular, intrathecal, or intrasternal injection, or infusion techniques. Topical administration may also involve the use of transdermal administration such as transdermal patches or iontophoresis devices. The compositions can be formulated in dosage unit formulations for administration comprising conventional nontoxic pharmaceutically acceptable adjuvants, carriers, excipients, and vehicles as described in Section I(b)(B)).

Actual dosage levels of active ingredients in a therapeutic composition of the disclosure may be varied so as to administer an amount of substituted phenethylamines that is effective to achieve the desired therapeutic response for a particular subject. A selected dosage level may depend upon a variety of factors, including the substituted phenethylamine in a composition, the activity of the therapeutic composition, formulation, the combination with other drugs or treatments, disease and longevity, the inflammation or inflammatory disorder, and the physical condition and prior medical history of the subject being treated. Determination of the proper dosage for a particular situation is within the skill of the practitioner.

In some aspects, pharmaceutical compositions comprise unit dose forms that contain an amount of substituted phenethylamines per dose. When the subject is a mouse, the unit dose can contain for example, but without limitation, about 5 μg of CBD. When the subject is a human, the unit dose can contain for example, but without limitation, about 0.75 mg of CBD.

In some aspects, a composition of the disclosure is administered as needed, upon development or shortly before development of symptoms. For instance, if the pulmonary inflammatory condition is asthma, the composition can be administered shortly before development of symptoms of asthma or upon development of symptoms of asthma or later.

In some aspects, the composition is administered regularly by following a prescribed treatment schedule. For instance, a composition of the instant disclosure can be administered routinely, at various intervals. For instance, compositions can be administered daily, weekly, monthly, or over several months. In some aspects, compositions are administered daily. In other aspects, compositions are administered weekly. In yet other aspects, compositions are administered monthly. Compositions can also be administered every three to six months. As it will be recognized in the art, the duration of treatment can and will vary and can be determined experimentally.

Administration of the compositions described herein can also be carried out as part of a treatment regimen that may include multiple instances of administration of one or more compositions comprising substituted phenethylamines. Such a regimen may be designed as a method of immediately treating a condition and/or as a method of long-term maintenance of the health of a subject after having been treated for a condition (e.g., prevention). For instance, a treatment regimen can be designed to delay the onset of the condition of interest in a subject. It will be appreciated that determination of appropriate treatment regimens is within the skill of practitioners in the art.

It will also be appreciated by those skilled in the art that a composition of the present disclosure may be co-administered with other therapeutic agents before, after, and/or during treatment with a composition of the disclosure. The term “co-administer” refers to administration of more than one active ingredient at the same time, just prior to, or just after the administration of one or more additional therapies. The compounds of the disclosure can be administered alone or can be co-administered to the patient. Co-administration is meant to include simultaneous or sequential administration of the compounds individually or in combination. Dosage amounts and intervals can be adjusted individually to provide levels of the administered compound effective for the particular clinical indication being treated. This will provide a therapeutic regimen that is commensurate with the severity of the individual's disease state.

### III. Kits

A further aspect of the present disclosure provides kits comprising one or more pharmaceutical compositions comprising a substituted phenethylamine detailed above in Section II. The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

The phrase “and/or,” as used herein, should be understood to mean “either or both” of the elements so conjoined, i.e., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of, ” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including”, “includes”, “having”, “has”, “with”, or variants thereof, are intended to be inclusive similar to the terms “comprising” and “comprises.”

The term “therapeutically effective amount” as used with reference to the present formulation(s) and/or component(s) thereof as described herein refers to the quantity of the formulation(s) and/or component(s) thereof necessary to render the desired therapeutic result. For example, an effective amount is a level effective to treat, cure, or alleviate the symptoms of a disorder for which the therapeutic formulation is being administered. Amounts effective for the particular therapeutic goal sought will depend upon a variety of factors including: the disorder being treated and its severity and/or stage of development/progression; the bioavailability and activity of the specific compound, biologic or pharmaceutical composition used; the route or method of administration and introduction site on the subject; the rate of clearance of the specific compound or biologic and other pharmacokinetic properties; the duration of treatment; inoculation regimen; drugs used in combination or coincident with the specific compound, biologic or composition; the age, body weight, sex, diet, physiology and general health of the subject being treated; and, like factors well known to one of skill in the relevant art. Some variation in dosage will necessarily occur depending upon the condition of the subject being treated, and the physician or other individual administering treatment will, in any event, determine the appropriate dosage for each individual patient.

As used herein, the term “administering” refers to providing a therapeutically effective amount of a formulation and/or components thereof to a patient via any of a number of potential delivery mechanisms including, but not limited to, oral, intravenous, transdermal, topical and/or inhalation, and the like. The formulation and/or components thereof of the present invention can be administered individually, but may also be administered with other compounds, excipients, fillers, binders, carriers or other vehicles selected based upon the chosen route of administration and standard pharmaceutical practice.

As used herein, the term “disorder” refers to a disorder, disease, condition, or other departure from healthy or normal biological activity, and can be used interchangeably. The condition may be caused by any of a number of physical factors. The condition may be caused by sporadic or inheritable genetic abnormalities. The condition may also be caused by non-genetic abnormalities. The condition may also be caused by injuries to a subject from environmental factors.

As used herein, the terms “treatment” or “treating” refers to arresting, inhibiting, correcting, or attempting to arrest or inhibit or correct, the existence, development, or progression of a disorder and/or causing, or attempting to cause, the reduction, suppression, regression, or remission of a disorder and/or a symptom thereof. As would be understood by those skilled in the art, various clinical and scientific methodologies and assays may be used to assess the development or progression of a disorder, and similarly, various clinical and scientific methodologies and assays may be used to assess the reduction, regression, or remission of a disorder or its symptoms.

As used herein, the term “treating” refers to: (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures. In the context of a neurodegenerative disorder, “treat” and “treating” encompass alleviating, ameliorating, delaying the onset of, inhibiting the progression of, or reducing the severity of one or more symptoms associated with the neurodegenerative disorder.

“Acute inflammatory conditions” as the term is used herein, and in accordance with normal medical parlance, refers to inflammatory conditions having a rapid onset and severe symptoms. The duration of the onset, from a normal condition of the patient to one in which symptoms of inflammation are seriously manifested, is anything up to about 72 hours. Acute inflammatory conditions are to be contrasted with chronic inflammatory conditions, which are inflammatory conditions of long duration, denoting a disease showing little change or of slow progression. The distinction between acute and chronic conditions is well known to those in the medical professions, even if they are not distinguishable by rigid, numbers-based definitions.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. 2C-H Exhibits Anti-Inflammatory Activity

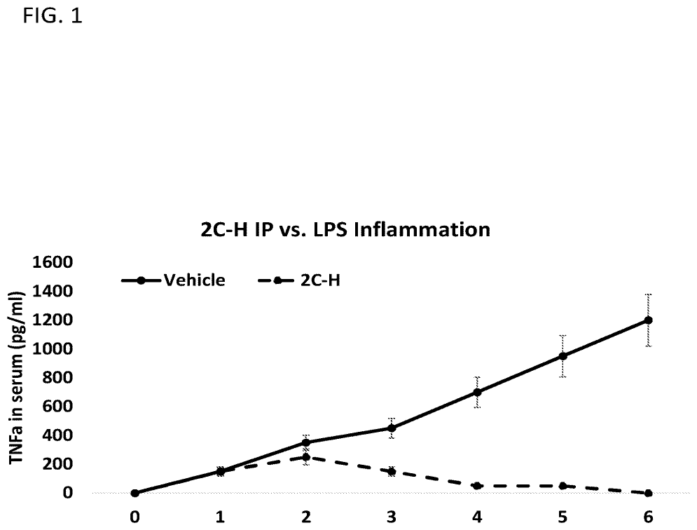

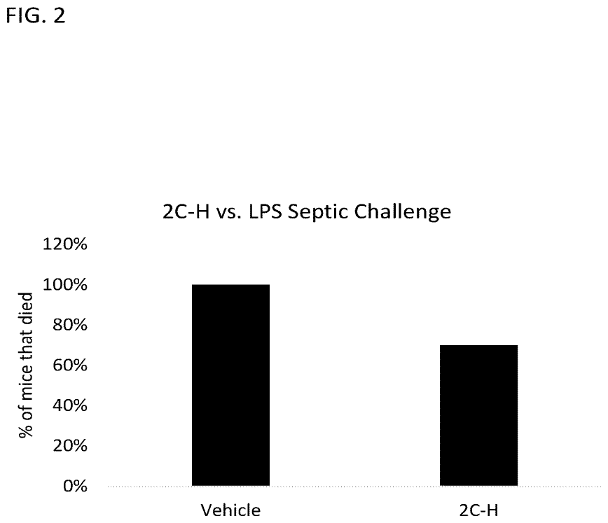

Mice were injected with LPS to initiate an inflammatory response (measured by TNFa in circulation) which was able to be quickly quelled by subcutaneous administration of 2C-H (FIG. 1). In another experiment, mice were also injected with a toxic level of LPS to initiate a lethal septic event, which was attenuated by administration of 2C-H (FIG. 2).

### Example 2. 2C-B Reduces Anxiety

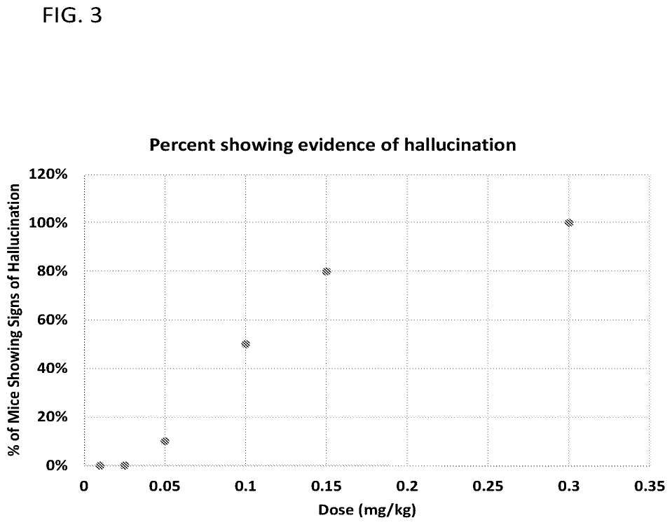

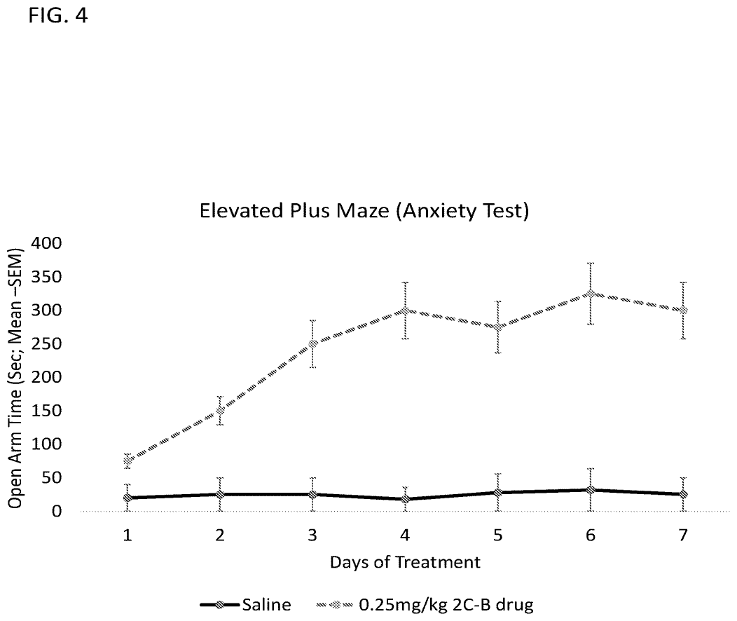

Mice were administered increasing amounts of 2C-B to identify sub-hallucinogenic doses (FIG. 3). It was determined that a dose of 0.025 mg/kg can be orally administered to mice without showing any signs of hallucination. Mice were then administered 2C-B for increasing amounts of time, then placed on the EPM apparatus for 5 minutes and time spent on the open arms obtained. As it can be seen in FIG. 4, the sub-hallucinogenic dose of 2C-B was capable of reducing anxiety in mice. Statistical analysis showed that non-hallucinogenic doses of 2C-B increased time spent on the open arm compared to saline. *p<0.001; **p<0.0001

What is claimed is:

1. A method of treating an inflammatory or neurological disorder in a subject in need thereof, the method comprising administering to the subject a therapeutically effective amount of a composition comprising a substituted phenethylamine.

2. The method of claim 1, wherein administering the composition comprises administering a unit dose of the composition to the subject, wherein the unit dose comprises a therapeutically effective amount of the substituted phenethylamine.

3. The method of any of the preceding claims, wherein the substituted phenethylamine is administered at a sub-hallucinogenic concentration.

4. The method of any of the preceding claims, wherein the substituted phenethylamine is a substituted phenethylamine of Table 1.

5. The method of any of the preceding claims, wherein the substituted phenethylamine is a member of the 2C-x family of substituted phenethylamines.

6. The method of any of the preceding claims, wherein the substituted phenethylamine is a member of the 2C-x family of substituted phenethylamines of Table 2.

7. The method of any of the preceding claims, wherein the substituted phenethylamine is 2C-H, 2C-I, 2C-B, or 2C-E.

8. The method of claim 1, wherein the method comprises treating inflammation.

9. The method of claim 8, wherein the inflammation is chronic inflammation or acute inflammation.

10. The method of claim 8, wherein the substituted phenethylamine is 2C-H or 2C-I.

11. The method of claim 8, wherein the inflammation is chronic inflammation and wherein the composition is administered in a unit dose comprising 2C-H at a concentration ranging from about 1 mg to about 100 mg, at a concentration of about 25 mg, at a concentration of about 50 mg, or at a concentration of about 100 mg.

12. The method of claim 8, wherein the inflammation is mild chronic inflammation and wherein the composition is administered in a unit dose comprising 2C-H at a concentration of about 25 mg.

13. The method of claim 8, wherein the inflammation is moderate chronic inflammation and wherein the composition is administered in a unit dose comprising 2C-H at a concentration of about 50 mg.

14. The method of claim 8, wherein the inflammation is severe chronic inflammation and wherein the composition is administered in a unit dose comprising 2C-H at a concentration of about 100 mg.

15. The method of claim 8, wherein the inflammation is acute inflammation and wherein the composition is administered in a unit dose comprising 2C-H at a concentration ranging from about 1 mg to about 100 mg, at a concentration ranging from about 100 mg to about 200 mg, at a concentration of about 25 mg, at a concentration of about 50 mg, at a concentration of about 100 mg, at a concentration of about 150 mg, or at a concentration of about 200 mg.

16. The method of claim 8, wherein the inflammation is mild acute inflammation and wherein the composition is administered in a unit dose comprising 2C-H at a concentration of about 25 mg.

17. The method of claim 8, wherein the inflammation is moderate acute inflammation and wherein the composition is administered in a unit dose comprising 2C-H at a concentration of about 50 mg.

18. The method of claim 8, wherein the inflammation is severe acute inflammation and wherein the composition is administered in a unit dose comprising 2C-H at a concentration of about 100 mg.

19. The method of claim 8, wherein the inflammation is systemic acute inflammation and wherein the composition is administered in a unit dose comprising 2C-H at a concentration of about 150 mg or at a concentration of about 200 mg.

20. The method of claim 8, wherein the therapeutically effective amount of 2C-H ranges from about 0.1 mg/kg to about 1 mg/kg body weight of the subject.

21. The method of claim 1, wherein the disorder is a psychological disorder.

22. The method of claim 20, wherein the substituted phenethylamine is 2C-B or 2C-E.

23. The method of claim 20, wherein the composition is administered in a unit dose comprising 2C-B at a concentration ranging from about 1 mg to about 10 mg, from about 1 mg to about 20 mg, from about 1 mg to about 100 mg, at about 10 mg, at about 15 mg, at about 20 mg, or at about 25 mg.

24. The method of claim 20, wherein the therapeutically effective amount of 2C-B ranges from about 0.1 mg/kg to about 1 mg/kg body weight of the subject.

25. A pharmaceutical composition for treating inflammation or a neurological disorder, the composition comprising a substituted phenethylamine.

26. The pharmaceutical composition of claim 25, wherein the substituted phenethylamine is a substituted phenethylamine of Table 1.

27. The pharmaceutical composition of any one of the preceding claims, wherein the substituted phenethylamine is a member of the 2C-x family of substituted phenethylamines.

28. The pharmaceutical composition of any one of the preceding claims, wherein the substituted phenethylamine is a member of the 2C-x family of substituted phenethylamines of Table 2.

29. The pharmaceutical composition of any one of the preceding claims, wherein the substituted phenethylamine is 2C-H, 2C-I, 2C-B, or 2C-E.

30. The pharmaceutical composition of any one of the preceding claims, wherein the substituted phenethylamine is 2C-H.

31. The pharmaceutical composition of claim 30, wherein the composition is in the form of a unit dose comprising 2C-H at a concentration ranging from about 1 mg to about 100 mg, at a concentration ranging from about 100 mg to about 200 mg, at a concentration of about 25 mg, at a concentration of about 50 mg, at a concentration of about 100 mg, at a concentration of about 150 mg, or at a concentration of about 200 mg.

32. The pharmaceutical composition of any one of the preceding claims, wherein the substituted phenethylamine is 2C-B.

33. The pharmaceutical composition of claim 32, wherein the composition is in the form of a unit dose comprising a sub-hallucinogenic dose of 2C-B.

34. The pharmaceutical composition of claim 24, wherein the composition is in the form of a unit dose comprising 2C-B at a concentration ranging from about 1 mg to about 10 mg, from about 1 mg to about 20 mg, from about 1 mg to about 100 mg, at about 10 mg, at about 15 mg, at about 20 mg, or at about 25 mg.

35. A kit for comprising one or more pharmaceutical compositions of claims 25 to 34 comprising a substituted phenethylamine.
